(19) United States
(12) Patent Application Publication
(10) Pub. No.: US 2010/0184024 A1
(43) Pub. Date: Jul. 22, 2010

(54) Method of Characterizing Nucleic Acids in a Mixed Sample

(75) Inventors: Christoph Gauer, Munchen (DE); Wolfgang Mann, Neudrossenfeld (DE)

Correspondence Address: Townsend and Townsend and Crew LLP, Two Embarcadero Center, 8th Floor, San Francisco, CA, 94111, US

(73) Assignee: BECKMAN COULTER, INC., Brea, CA (US)

(21) Appl. No.: 12/225,774

(22) PCT Filed: Feb. 15, 2007
(86) PCT No.: PCT/EP2007/001328

§ 371 Date: Mar. 24, 2010

(30) Foreign Application Priority Data
Mar. 27, 2006 (DE) 10 2006 014 000.1

(51) Int. Cl. C12Q 1/68

(52) U.S. Cl. 435/6

(57) ABSTRACT

A method is provided for characterizing a mixed sample having at least two particles with nucleic acids from different individuals, where each particle has nucleic acid from one or more individuals, in particular for the quantitative determination of the absolute and/or relative copy number of a predetermined sequence of an individual, of which nucleic acid is present in the mixed sample, having the steps:
a) isolating the particles and applying at least two individual particles to a substrate, where each of the at least two particles is deposited in each case individually to a hydrophilic reaction site, surrounded by a hydrophobic zone, of the substrate in a volume of less than 10 μl so that precisely one particle is present per reaction site,
b) analysis of at least two of the particles deposited to the substrate at the reaction site of the substrate to assign each of the particles to individuals from the mixed sample by genotyping, where at least 80% of the particles analysed are to be assigned to an individual, and
c) further characterization of the analysed particles. Moreover, a kit which is suitable in particular for carrying out this method, is also provided.

The present invention relates to a method for the characterization of a mixed sample containing at least two particles with nucleic acids of different individuals, with each particle including nucleic acid of one or more individuals, in particular for the quantitative determination of the absolute and/or relative number of particles with the nucleic acid of an individual present in a mixed sample and/or for the determination of the genotype of one or more individuals from a mixed sample, in particular for the quantitative determination of the absolute and/or relative copy number of a predetermined sequence of an individual from the nucleic acid contained in the mixed sample. Furthermore, the present invention relates to a kit for the determination of the genotype of one or more individuals from a mixed sample which contains particles with nucleic acids of different individuals, which is in particular suitable for the quantitative determination of the absolute and/or relative copy number of a predetermined sequence of an individual from the nucleic acid which is contained in the mixed sample.

The need arises in a multitude of technical fields to characterize mixed samples i.e. biological samples containing nucleic acids of different individuals in order to draw conclusions on the identity and/or one or more specific genotype features of one or more of the individuals whose nucleic acid(s) is or are contained in a mixed sample. Simply for example applications in forensics, in gene technology, for example in the context of cloning or in medical diagnostics are named.

In forensics, samples from a site of a crime are frequently only available which contain nucleic acids of two or more different persons in order to draw conclusions from the mixed samples which permit recognitions concerning the identity of the perpetrator. As a rule it is generally not known how the mixed sample is composed, and in particular from how many different individuals nucleic acid is contained in the mixed sample and how the quantity ratio of the nucleic acids of the single individuals in the mixed sample is relative to one another.

In medical diagnostics the task also frequently arises of characterizing a mixed sample. As an alternative to amniocentesis or choriozottic biopsies, which involve risks of infection for the pregnant woman who is investigated, it has been attempted in recent time, in prenatal diagnostics, to determine the genotype of a foetus from maternal blood containing foetal cells in order to be able to recognize serious conditions already in the prenatal stage. Since many of the partly serious conditions occurs by deviations from the normal copy number of nucleic acid sequences in the genome, such conditions can already be reliable diagnosed at an early stage of the development by determination of the copy number of certain chromosomes or of certain gene sections. Examples for partly serious anomalies which can be attributed to an increased copy number of whole chromosomes are Trisomie 18 (Edward's syndrome), Trisomie 13 (Patau syndrome) and also Trisomie 21 (Down syndrome). In each of these conditions the copy number of the corresponding chromosome 18, 13 and 21 per cell is three, whereas healthy individuals only have two copies of the chromosomes per cell. In all three cases the increase of the copy number of the relevant chromosome leads to the most serious developmental problems. In addition to conditions, which are to be attributed to an increased copy number of whole chromosomes, a multitude of conditions are known which relate to a changed copy number of genes or gene sections. Simply by way of example the Huntington disease should be named in this connection; a progressively developing neuro-degenerative condition characterized by abnormal involuntary movements with an increasing decay of the mental and physical abilities. By the determination of the copy number of the corresponding chromosome, gene or gene sections in the foetal cells a corresponding condition of this kind can already be diagnosed prenatally. However, the determination of the genotype of the foetus from maternal blood is problematic since foetal cells in maternal blood only arise in a frequency of about 1:1,000,000 (foetal cells/maternal cells) and the precise relative ratio between maternal cells and foetal cells is not initially known and cannot be found without further complicated and costly investigations.

A similar problematic arises in the diagnosis of cancer, for example in investigation of a body sample for the presence of cancerous cells. If the body sample actually contains cancerous cells then this is a mixed sample containing healthy cells and cancerous cells (which are regarded in the context of the present invention as cells of two different individuals), with the quantity ratio of the individual cell types relative to one another not being known and having to be determined with complicated and expensive investigations in order to determine at what stage of advance the cancer is.

As a result of the presence of nucleic acids of different individuals and of the unknown quantity ratio of these different nucleic acids relative to one another a direct genetic investigation of mixed samples is frequently not possible or leads to false results since the nucleic acid(s) of other individuals contained in addition to the nucleic acid of the individual to be characterized disturb the characterization of the nucleic acid of the individual to be characterized. This is in particular the case when the quantity of the nucleic acid of the individual to be characterized in the mixed sample is significantly less than the quantity of the nucleic acid present alongside of it of another individual, such as in the case of maternal blood containing foetal cells. If the mixed sample is enriched with respect to the individual to be characterized, for example by enriching the foetal cells by means of fluorescence marked anti-bodies, then one does not as a rule succeed in obtaining a pure sample with respect to the individual to be characterized. In the known methods false positive results arise (a maternal cell is incorrectly typed as a foetal cell) and/or false negative results (a foetal cell is overlooked).

The above-named problem also results with individual cell investigations when the corresponding cell was not cleanly isolated and another cell or other cells attached to it is/are present unrecognized in the isolate. During the isolation of individual cells from tissue an undesired contamination of the individual cells with the nucleic acid of other cells frequently arises because nucleic acid containing cell membrane fragments of other cells are frequently deposited on the target cell. The results obtained in the characterization of such a cell isolate are frequently incorrect as a result of the background of other cells that is present (Fendt & Raffeld, J. Clinical Pathology (2000), 53: 666-672).

This can be explained with respect to the following conceptual experiment: It is intended to determine with respect to a body sample of a patient whether the cells have mutated to cancerous cells. In this respect it is known that a cancerous cell over-expresses a certain gene which is why the cancerous cell to be detected has a double copy number of mRNA of the said gene relative to the healthy cell. If one now investigates a mixed sample which for example consists of a cancerous cell to which a healthy cell adheres one obtains a mixed response which is composed of the sum of the expression of the corresponding gene of the healthy cell and the expression of the corresponding gene of the mutated cell. The gene expression, which can be quantified by the mRNA content, is measured in comparison to the healthy cell. In this respect the corresponding gene expression of a healthy cell is 100%, that of 2 healthy cells is 200% and that of 3 healthy cells is 300% whereas the gene expression of a cancerous cell amounts to 200%. Thus the investigation of the mixed sample consisting of a healthy cell and a cancerous cell leads to a gene expression of 300% (100% for the healthy cell plus 200% for the cancerous cell) which in relationship to a reference consisting of two healthy cells with a gene expression of (100%+100%=) 200% leads to a ratio sample/reference of 300/200=1.5. Had one in contrast carried out two experiments with individual cells free of cell components of other cells, then one would have obtained for an investigated healthy cell (100% gene expression) a ratio sample to reference (1 healthy cell with 100% gene expression) of 1. In the investigation of a cancerous cell (200% gene expression) one would in contrast have obtained a ratio of a sample to reference (1 healthy cell with 100% gene expression) of 2 which in comparison to the ratio of 1.5 for the two cell experiment is significantly easier to detect.

However, even if pure individual cells free of foreign nucleic acid are presented for an individual cell investigation statistically seen only 40 to 70% of the cases result in an amplification product with a PCR carried out on it, because individual cells in customary micro-titre plates are frequently deposited unrecognized at the edge of the container and thus are not suspended after addition of the reaction solution in a volume of smaller than 50 μl typical for the PCR. In a 96 well micro-titre plate, in which precisely one cell was deposited per well with customary pipetting devices, amplification reactions carried out are thus only obtained for approximately 35 to 65 percent of the samples with the customary method.

There are currently no methods with which a mixed sample containing nucleic acids of different individuals for which the qualitative and/or quantitative composition, i.e. from which the number of the different individuals from whom nucleic acid is contained in the mixed sample and/or the quantity ratio of the individual different nucleic acids relative to one another is unknown, can be reliably and rapidly analyzed with respect to the genotype of an individual contained in the mixed sample. Rather, the known methods for this purpose lead, as a result of the background of nucleic acids of the other individuals different from the individual to be investigated, to incorrect or at least to unreliable results. Moreover these methods are mainly time-consuming and cost intensive.

The object of the present invention is thus to make available a method for the characterization of a mixed sample containing at least two particles with nucleic acids of different individuals with which the absolute number and/or the relative number of particles present in the mixed sample with nucleic acid of an individual and/or the genotype of one or more individuals can be determined simply, rapidly and in particular reliably from the mixed sample.

In accordance with the invention this object is satisfied by a method in accordance with patent claim 1 and in particular by a method for the characterization of a mixed sample containing at least two particles with nucleic acids of different individuals, in particular for the quantitative determination of the absolute and/or relative number of particles with nucleic acid of an individual present in a mixed sample and/or for the determination of the genotype of one or more individuals from a mixed sample, in particular for the quantitative determination of the absolute and/or relative copy number of a predetermined sequence of an individual from whom nucleic acid is contained in the mixed sample, wherein each particle includes nucleic acids of one or more individuals, including the steps:
a) separation of the particles and the application of at least two individual particles onto a substrate, with each of the at least two particles being respectively individually deposited onto a hydrophilic reaction site of the substrate in a volume of less than 10 with each hydrophilic reaction site being surrounded by a hydrophobic region, so that precisely one particle is present for each reaction site,
b) investigation of at least two of the particles deposited on the substrate on the reaction site of the substrate by means of an amplification reaction in order to respectively associate the particles with individuals from the mixed sample by genotyping wherein at least 80% of the investigated particles of an individual are associated with the different individuals and
c) further characterization of the investigated particles.

In the sense of the present invention a mixed sample will be understood to be a sample, in particular a biological sample which includes at least two particles respectively containing nucleic acid, with nucleic acids of at least two different individuals being contained in the sample either per particle or in the totality of the particles. The term particle signifies in this connection a small fragment. The nucleic acid can be contained in the particle or bound to the particle. Examples for corresponding particles are cells, in particular unlysated cells with nucleic acid contained therein and magnetic particles with nucleic acid which is for example bound via a hybridisation to a primer.

The term individual includes, in the sense of the present invention, not only a person different from others—in the case of humans—but rather in particular also different cell types of a person which are distinguished from one another with respect to their genotype. Examples for this are genetic mosaics or chimeras, i.e. cells of different genotype of a person which first form by mixing or exchange of different genotypes (chimeras) or arise in an individual (genetic mosaic). An example for a genetic mosaic are cancerous cells which have arisen through LOH (“loss of heterozygosity”).

A method for the characterization of a mixed sample signifies in the context of the present invention in particular that a mixed sample is characterized qualitatively and/or quantitatively with respect to its composition. A characterization of a mixed sample thus includes for example the quantitative determination of the absolute number of different individuals present in a mixed sample, the quantitative determination of the relative number/frequency of an individual in the mixed sample (for example the determination of the proportion by percent of a cell type A in a biological sample including the cell types A and B) and/or the determination of the genotype of one or more individuals represented in a mixed sample.

In the sense of the present invention the determination of the genotype of one or more individuals will in particular be understood to mean the characterization of at least one predetermined sequence of an individual with respect to the presence or absence, copy number and/or nucleic acid sequence, i.e. in particular the determination of the absolute or relative number of a predetermined sequence, for example of a genome, of a gene or of a gene section.

Furthermore, the relative quantitative determination of the number of a predetermined sequence in an individual in the sense of the present invention signifies the determination of whether the genome of an individual contains fewer than, equal or more copies of a predetermined sequence than that of a reference sample and absolute quantitative determination of the number of a predetermined sequence in an individual signifies in the sense of the present invention the determination as to which specific number of copies of the predetermined sequence is present in the genome of the individual.

Moreover, the term homologous sequence designates, in the sense of the present invention, sequences which have a similarity with respect to their nucleotide sequence of at least 70%, preferably of at least 80% and particularly preferably of at least 90% and especially preferably of at least 95%, whereas non-homologous sequences are those which have a correspondingly lower sequence similarity amongst one another.

Using the method of the invention a mixed sample containing at least two particles with nucleic acids of different individuals can be characterized rapidly simply and in particular reliably. Using this method, reliable results can in particular be obtained with respect to the absolute and the relative number of particles with nucleic acid of an individual present in a mixed sample. Furthermore this permits the reliable determination of the genotype of one or more individuals from the mixed sample.

An important feature of the method of the invention is that the particles or cells of a mixed sample are first separated in step a) in such a way that a later deposition of precisely one particle or one cell is possible per reaction site of the substrate which, in contrast to many individual processes known from the prior art, is free from other cells or components of other cells bound thereto. In this way it is ensured that this cell or this particle can be analyzed without a background of nucleic acid foreign to the individual.

Moreover, through the deposition of in each case one cell on the hydrophilic reaction site of the substrate surrounded by the hydrophobic region, the formation of a liquid droplet formed from a liquid contained in the cell isolate or added from the cell after the deposition on the reaction site is made possible which sticks comparatively firmly to the substrate so that the subsequent steps b) and c) of the method of the invention can be carried out directly at the reaction site is made possible without the cell having to be transferred into a closed reaction vessel or the like. In this way complicated and time-consuming transfer steps are avoided on the one hand. Furthermore, it is possible in this way that a corresponding number of plural probes can be prepared in parallel on hydrophilic reaction sites spatially separate from one another comprised on the substrate without the danger existing the liquid drops which lie spatially close together mix with one another with minor shaking or as a result of the running of liquid drops as a consequence of a drop volume which is too high.

In particular the advantage of the deposition of particles on such a substrate provided in accordance with the invention, in contrast to the conventional micro-titration plate, lies in the fact that an optical control of the material to be analyzed is possible directly prior to the actual analysis. For example, it can be determined unambiguously by microscope that precisely only one single cell was deposited on each reaction site. This is not possible in a 3-dimensional reaction vessel, as a result of lack of depth of sharpness of the microscope and for other reasons, without considerable cost and complexity. Thus, in combination with the optimization of the genotyping in step b), which for example takes place via PCR, it can be ensured that at least 80% of the particles investigated can be associated with an individual or are associated with an individual.

By introducing the cell in a volume of preferably less than 1 μl on the reaction site the method steps b) and c) can be executed directly at the reaction site without the sample previously having to be concentrated by evaporation or transferred into a closed reaction vessel. Furthermore, through the minimal liquid volume remaining at the cell after the separation, the deposition of larger quantities of contaminants at the reaction site which potentially disturb the subsequent method steps b) and c), is prevented in particular when these method steps includes an enzyme reaction. In distinction to this, in many of the known individual cell processes the cells are isolated from cell culture medium or body fluid such as blood or the like, with the cell being deposited in a considerable volume of cell culture medium or body fluid into a reaction vessel. As a result of the significant quantities of contaminants contained in this volume of cell culture medium or body fluid an enzymatic reaction is not possible with this process without further time-consuming and work-intensive cleaning of the sample.

For the above reason it is preferred for the at least two individual particles to each be deposited in a volume of less than 100 nl, particularly preferably less than 10 nl and especially preferably less than 1 nl and most preferred less than 100 pl on the corresponding reaction site of the substrate.

A further important feature of the method of the invention is the association of the at least two cells or particles individually deposited on the reaction sites to an individual contained in the mixed sample by a determination carried out on the reaction site of the substrate from which the deposited particles of the individuals represented in the mixed sample contain nucleic acid, with at least 80% of the investigated particles being able to be associated with an individual or being associated with an individual. In this way, prior to the further characterization of the investigated particles in accordance with method step c) it is on the one hand verified that the cells deposited on the reaction sites are actually pure individual cells free from components of cells of foreign individuals. On the one hand, it can be thereby verified whether each of the deposited cells is a target cell or a false positive cell. Consequently, the results of a further characterization of the investigated cell carried out following this can be uniquely associated with an individual. As a result of the separation and the subsequent association of the deposited particles to an individual contained in the mixed sample by genetic analysis an unambiguous conclusion can be drawn in this way.

During the investigation by genotyping in the method step b) preferably at least 85%, in particular preferably at least 90%, particularly preferably at least 95% and especially preferably 98% and most preferably 100% of the investigated particles can be associated with an individual.

In accordance with a preferred embodiment of the present invention the further characterization of the investigated particles in accordance with method step c) includes the determination of the absolute and/or relative number of particles present in the mixed sample with nucleic acid of an individual and/or the determination of the genotype of the particle deposited on the reaction site of the substrate. In this way the results relating to the quantitative and/or qualitative composition of the mixed sample are obtained. In this embodiment it can, for example, be determined from maternal blood containing foetal cells whether the foetus has Trisomie 21 or not. As an alternative to this, the progression of cancer in a patient can be determined with this embodiment in that the proportion in percent of cancerous cells in a cancer tissue, i.e. in a mixed sample containing cancer cells and healthy body cells is determined.

Preferably the particles deposited on the reaction sites of the substrate are cells, in particular preferably unlysated cells. The latter is preferred because, with an unlysated cell in distinction to a lysated cell, it can be ensured by optical control that this includes the whole genome of an individual. Alternatively to this, the particles can however in each case be every fragment having nucleic acid of a specific individual, such as for example a fragment marked with a DNA or RNA probe, with DNA or RNA hybridised onto the probe.

As a further development of the concept of the invention it is proposed to carry out the separation and/or the application of the at least two particles onto the substrate by means of a capillary, by means of laser pressure catapulting technology (“Laser Pressure Catapulting”-technique) or by means of a through-flow cytometer preferably by means of a fluorescence-activated cell sorter (FACS). In the context of the present invention it was found that using each of the above-named techniques individual cells can be prepared from the mixed sample intentionally free of components of other cells and can be deposited on a substrate. This is advantageous because the particle or the cell has only nucleic acid of one individual and thus can be genetically investigated without a background of a foreign nucleic acid. A further advantage of the above-named method is that using it the particles or cells can be deposited in an extremely small liquid volume on the substrate and can thus be investigated directly and enzymatically, i.e. with a further cleaning. In distinction to this, in the customary processes for preparation of an individual cell, for example a micromanipulation, in which the individual cell is sucked with a capillary from a highly diluted suspension using a microscope, the cells are isolated with considerable quantities of liquid. As a result of the contaminants contained in the liquid, for example cell culture medium or blood such as proteases, nucleases, salts and the like such isolates require a removal not only of the liquid but also of the contaminants before the so isolated cell can be used in an enzymatic reaction.

Examples of commercially obtainable apparatus which utilize one of the above-named techniques are the manual capillary system for example of the company Eppendorf, Hamburg, the automatic system CellCelector of the company AVISO Gmbh, Gera, apparatuses based on the Laser Pressure Catapulting technique, for example of the company PALM, Bernried and FACS apparatuses for example from the companies Becton Dickinson and Dako Cytomation.

The manner of operation of FACS apparatuses is normally as follows: A liquid suspension containing the particles or cells is led through a nozzle at which the liquid flow is split up into individual liquid droplets separated from one another with the individual liquid droplets each containing a predetermined number of cells, all liquid drops or selectively individual liquid drops are electrically charged after the separation from the nozzle and the individual liquid droplets are guided through an electric field whereby one or more electrically charged drops are selectively directed onto a substrate. On guiding the individual liquid drops through the electric field only the electrical charged drops or droplets is/are deflected and applied onto the correspondingly positioned substrate. The separation of the liquid suspension at the nozzle takes place by a pieco-electric modulation in which a periodic pressure fluctuation is exerted on the liquid jet flowing through the nozzle as a result of which liquid drops form at a nozzle with a defined and reproducible size and these tear away from the liquid jet. By corresponding setting of the concentration of the cells in the liquid suspension, the speed of flow of the suspension and corresponding adjustment of the pieco-electric modulation a situation can be achieved in which each liquid drop has a defined and reproducible size and contains a predetermined number of cells, for example precisely one cell. The separation of the droplets from the nozzle takes place as a result of the impulse of the pressure fluctuations assisted by gravity.

The substrate used in the method of the invention is preferably an object carrier, particularly preferably a glass object carrier, on the one hand because these are flat and, on the other hand, because these are excellently suited to the application of hydrophilic regions (here also termed reaction sites) and hydrophobic regions.

In order to enable subsequent enzymatic reactions in the liquid drops deposited on the substrate it is proposed in a further development of the concept of the invention to make the hydrophilic reaction sites on the substrate substantially circular and to surround these by an at least substantially circular ring-shaped hydrophobic region. In order to obtain a symmetric arrangement the circular ring-shaped hydrophobic region should preferably concentrically surround the circular-shaped hydrophilic regions.

An even better formation of liquid drops on the substrate is achieved when the hydrophobic region surrounding the hydrophilic reaction site of the substrate is surrounded at its outer side by a hydrophilic region, which is preferably essentially of circular ring-shape and particularly preferably concentrically surrounds the hydrophobic regions. The outer hydrophobic circular ring is preferably surrounded at the outer side by a hydrophobic region. Thus a particularly preferred arrangement consists of a hydrophilic region concentrically surrounded by two circular rings, with the inner of the two circular rings being hydrophobic and the outer of the two circular rings being hydrophilic and with the outer hydrophilic ring being surrounded at the outer side by a hydrophobic region.

Particularly good results are in particular obtained when the hydrophilicity of the hydrophilic reaction site and the hydrophobicity of the region surrounding it are set such that on applying less than 10 μl water to the reaction site a water droplet with a contact angle of 20 to 70°, preferably from 30 to 60° and particularly preferably from 40 to 50° is formed. In this way it is ensured that a stable liquid drop forms which sticks firmly to the reaction site so that the liquid drop does not separate from the glass plate or run on the glass plate even with the smallest vibrations of the substrate, such as arise during transport of the substrate, for example in a laboratory.

The diameter of the hydrophilic reaction site preferably amounts to between 0.3 and 3 mm insofar as it is, as preferred, of substantially circular shape.

In order to enable the parallel preparation of a plurality of samples it is proposed in accordance with a further development of the concept of the invention to provide from 2 to 1.000, preferably 12 to 256, particularly preferably 24 to 96 and especially preferably 48 different essentially circular hydrophilic reaction sites on the substrate which are each concentrically surrounded by a substantially circular hydrophobic region which is surrounded at the outer side by a substantially circular ring-shaped hydrophilic region which is preferably adjoined at the outer side by a hydrophobic region again.

The method of the invention is basically suitable for the characterization of all mixed samples independent of the nature of the particles that are used and independent of the number of the different individuals represented in mixed sample. Good results are in particular obtained when the mixed samples nucleic acid of at least two but less than 10 different individuals, particularly preferably of at least two but less than or equal to 5 different individuals, particularly preferably of two or three different individuals and most preferred of precisely two different individuals.

The method in accordance with the invention can basically be used for all mixed samples, independently of the concentration differences of the individual nucleic acids relative to one another. Good results are in particular obtained when the difference in concentration of the nucleic acids relative to one another of the single individuals contained in mixed sample amounts to between 1:1,000 and 1:1, preferably to between 1:100 and 1:1 and particularly preferably to between 1:10 and 1:1.

However, if the proportion of the nucleic acid of the individual to be investigated at the mixed sample amounts to less than 1:1,000 relative to the nucleic acids of the other individuals, such as for example is regularly the case with maternal blood containing foetal cells, it has proven advantageous to enrich the mixed sample prior to the separation and prior to the application onto the substrate in accordance with step a) with respect to the particles with the nucleic acid of the individual to be investigated, because otherwise a large number of particles has to be investigated until statistically a target particle of the individual to be investigated has been applied onto the substrate. The enrichment can take place in every manner known to the person skilled in the art, for example by means of fluorescence-marked anti-bodies which specifically bind to the cell type which is to be enriched and thus mark it. Alternatively to this, coated catching particles or coated magnetic particles can be used. A further example for a suitable enrichment method is the use of a through-flow cytometer, in particular of a fluorescence activated cell sorter (“FACS) which, as a rule, operates with fluorescence-marked anti-bodies for the classification of particles and/or their enrichment. The apparatus offers the advantage during the enrichment of the particles or cell species to be enriched that the fluorescence-recognition and the enrichment are united in one apparatus.

As a result of the above-named characteristics the method of the invention is in particular suitable for the characterization of mixed samples which include maternal blood containing foetal cells and preferably for the characterization of mixed samples consisting of foetal cells containing maternal blood.

In the same way the method of the invention is pre-destined for the characterization of a mixed sample containing healthy cells and also cancer cells characterized by LOH and preferably for a mixed sample consisting of healthy cells and also cancerous cells characterized by LOH.

Moreover, the method of the invention has proved to be served to be just as well suited for the characterization of a mixed sample containing healthy cells and also cancer cells characterized by MIN (micro-satellite instability) or preferably of a mixed sample consisting of healthy cells and also cancerous cells characterized by MIN.

In accordance with the invention, after the separating of the particles and the application of at least two individual particles onto respectively one hydrophilic reaction site of the substrate surrounded by a hydrophobic region in a volume of less than 10 the particle deposited on each individual reaction site of the substrate is associated in accordance with method step b) with an individual represented in the mixed sample and the particles investigated in the method step b) are subsequently further characterized in accordance with the method step c). The association of the particles to an individual contained in the mixed sample in accordance with step b) preferably takes place by means of an amplification reaction, with primer pairs for gene sections specific for the target individual suitably being used in the amplification reaction.

The further characterization of the investigated particles can for example relate to the determination of the absolute number of particles present in the mixed sample containing nucleic acid of an individual or to the determination of the relative proportion of the particles containing nucleic acid of an individual related to the total mixed sample. In the same way the further characterization of the mixed sample can be the determination of the relative or absolute copy number of a Chromosome, of a gene or of a gene section. In the last-named case in particular it is preferred that the further characterization of the particles in accordance with step c) also takes place on the reaction site of the substrate by means of an amplification reaction.

The amplification reaction can be a reaction known to everyone skilled in the art with which nucleic acids, be it DNA or a RNA, can be multiplied, preferably almost exponentially multiplied. In particular, the carrying out of a polymerase chain reaction (PCR) as an amplification reaction has proved advantageous.

Independently of the nature of the amplification reaction which is carried out, it has proved advantageous, in particular in the case in which the particles are cells, to solubilize the particles deposited on reaction sites thermally or by at least one freezing/thawing cycle carrying out the amplification reaction.

The amplification reaction which is carried out is advantageously a specific amplification reaction.

In particular in those cases in which the particles in the mixed sample contain extremely little nucleic acid, for example less than 1 pg, which can for example arise in the case that magnetic particles with nucleic acid hydrolysed via probes present on the surface are used as particles, it has proved advantageous to multiply the nucleic acids contained in or on the particles by an unspecific PCR prior to the amplification reaction for the association of the particles to an individual contained in the mixed sample in accordance with step b) and/or prior to the amplification reaction for the further characterization of the investigated particle in accordance with step c). After the unspecific PCR a specific PCR can take place.

In accordance with the invention at least two particles which are respectively individually deposited on a respective reaction site on the substrate in accordance with step b) are investigated in order to associate these by genotyping on the reaction sites of the substrate with individuals from the mixed sample. For this purpose the embodiments described in the following have proved to be particularly suitable.

In accordance with a preferred embodiment of the present invention the reaction components necessary for the carrying out of the amplification reaction are presented on the hydrophilic reaction site, in the case of a PCR preferably the primers, before the particle is deposited on the reaction site. It is however also possible to apply the reaction components on the hydrophilic reaction site of the substrate in the form of liquid onto the particle after deposition of the particle.

In a further development of the concept of the invention it is proposed to adapt the amplification reaction to amplify one sequence or at least two sequences which are homogeneous to one another and/or not homogeneous from the coded DNA range and in particular preferably from the non-coded DNA range. In known manner the non-coded DNA range is substantially more polymorphous than the coded DNA range so that, by amplification of sequences from the non-coded DNA range, individual specific sequences can be amplified with a relatively large probability. This is both advantageous with forensic mixed samples as also in the characterization of the genotype of foetal cells from maternal blood containing foetal cells.

For the same reason it has proved advantageous to adapt the amplification reaction to the amplification of one sequence or of at least two highly polymorphous sequences which are homogeneous to another and/or not homogeneous. In particular in cases in which the amplification reaction is adapted to amplify a sequence or at least two sequences which are homogeneous to one another and/or not homogeneous, good results are obtained for sequences which are selected from the group consisting of STR sequences, VNTR sequences, SNP sequences and any desired combinations hereof. STR or short tandem repeat sequences are highly polymorphous sequences which consist of only two to four by long repetition units and have a high variability between the single individuals. In distinction to this VNTR or variable number of tandem repeat sequences consist of repetitive DNA sections built up from approximately 15 to 30 by length, the total length of which is determined by the number of repetitions of this base unit. VNTR sequences are as a rule highly polymorphous, i.e. the number of the respective repetition units is distinguished greatly between the different individuals. For SNP's (single nucleotide polymorphism) these are the simplest polymorphisms in which the homologous sequences are only respectively distinguished by a base. These sequences are also excellently suited for the carrying out of the method of the invention since these are very strongly distinguished between the single individuals. Apart from this all other highly polymorphous sequences are however also suitable as markers for the method in accordance with the invention.

Furthermore, it is preferred that the amplification reaction or in particular an amplification reaction used in step c) is adapted to amplify one or at least two sequences which are homologous to one another and/or not homologous to one another, which only arise once per allele in the genome of the individual. Thus, in the characterization of the amplification products, conclusions can be drawn on the individual alleles of an individual so that, for example, a number of individual alleles of an individual in a mixed sample can be determined.

In accordance with a further preferred embodiment of the present invention the investigation in accordance with step b) and the further characterization of the investigated particles in accordance with step c) take place simultaneously, i.e. in one method step.

The amplification reaction is preferably adapted to amplify between 1 and 100, preferably between 2 and 20 and particularly preferably between 5 and 15 sequences which are homologous to one another and/or not homologous of the individual mixed sample to be sought. In this way sufficient different amplification products are obtained in order to obtain targeted individual specific results during the characterization of the amplification products. On the other hand, the experimental cost and complexity is not yet too large.

For the further characterization of the investigated particles in accordance with the method step c) the number of the different amplification products obtained during the amplification reaction can, for example, be determined, with the determination of the number preferably including the determination of the presence or absence of at least one amplification product and also the determination of a second physically and/or chemically measurable parameter of the amplification products that are obtained. For the determination of the presence or absence of amplification products all methods known to the person skilled in the art for this purpose can be used, with for example gel electrophoresis, familiar hybridisation techniques, in particular those on a DNA array being named by way of example. In this connection it can be expedient, in dependence on the detection method that is used, to define threshold values above which the presence of a PCR product and below which the absence of a PCR product is assumed.

In order to check the correct running of the amplification reaction, and in particular to find faults with the thermo cycler that is used at an early time, it is proposed, as a further development of the concept of the invention, to carry out in parallel to the amplification reaction an amplification reaction under same conditions with a control sample which, with correct running of the PCR leads to a known number of amplification products with a known length.

For the further characterization of the particles that are investigated in accordance with method step c) it has, moreover, proved advantageous to set the parameters in the amplification reaction in such a way that the relative frequency for a positive amplification reaction for the sequences which are to be amplified and which are homologous to one another and/or not homologous is respectively at least substantially of the same level. It is thus reliably precluded that only individual amplification products are obtained and others not obtained as a result of any irregularities in the carrying out of the amplification reaction, which could lead to a false result during the analysis. Good results are obtained, in particular, when the parameters in the amplification reaction are selected such that the relative frequency for a positive amplification reaction for each of the sequences which are homologous to one another and/or not homologous to one another amounts to between 0.2 and less than 1, preferably to between 0.4 and 0.6 and also particularly preferably to approximately 0.5.

For the characterization of the amplification products, in particular in cases in which the relative copy number of a predetermined sequence of an individual represented in the mixed sample is to be determined then, prior to, after or preferably in parallel with the amplification reaction for the at least two particles to be investigated, at least one amplification reaction should be carried out under the same conditions with a reference sample under the same conditions as used for the at least two particles deposited from the mixed sample on one reaction site in each case, with the reference sample preferably having the same quantity of nucleic acid as the deposited particles and the reference sample preferably having a known genotype. From the comparison of the number of the different amplification products obtained with this at least one amplification reaction with the number of different amplification products obtained with the amplification reactions carried out with the deposited particles, the relative copy number of the investigated predetermined sequence of the investigated individual can be determined.

For the cases in which the absolute copy number of the predetermined sequence of the individual to be investigated is to be determined then is proposed, in a further development of the context of the invention to compare the number of different amplification products obtained with the amplification reaction(s) carried out with the at least two deposited particles with at least one frequency distribution. A frequency distribution of this kind is preferably obtained by separate respective multiple carrying out of the same amplification reaction and under the same reaction conditions as used for the at least two particles deposited from the mixed sample on the reaction sites using at least two different reference samples, with the same quantity of nucleic acid as contained in the particles being used in the amplification reactions and the at least two different reference samples each having a known copy number different from one another of the predetermined sequence. In this connection the amplification reactions for the reaction samples can be carried out prior to, after or—particularly preferably—in parallel to the amplification reaction for the particles to be investigated. By subsequent determination of the number of different amplification products obtained per reference sample and by comparison of these numbers with the numbers of different amplification products obtained with the amplification reactions carried out for the particles deposited on the reaction sites of the substrate, the absolute copy number of a predetermined sequence of the individual to be investigated or of the individuals to be investigated can be determined.

A frequency distribution is preferably used for the recording of which the amplification reaction carried out for each of the least two reference samples was carried out multiply, for example ten times or one hundred times. Since starting material with a known copy number of the predetermined sequence is used in the amplification reactions for the recording of the frequency distribution, a conclusion can be reliably drawn from this comparison concerning the number of copies of the predetermined sequence in the particle of the mixed sample to be investigated.

As an alternative to the carrying out of an amplification reaction for the association of the at least two particles to be investigated to an individual contained in the mixed sample by a genotyping carried out on the reaction site of the substrate in accordance with method step b) and/or for the further characterization of the particles investigated in accordance with method step c), the association of the particles investigated to an individual contained in the mixed sample and/or the further characterization of the particles investigated on the reaction site of the substrate can also take place by a gene expression investigation at the mRNA level.

A further subject of the present invention is a method for the characterization of a mixed sample containing at least two particles with nucleic acids of different individuals, with each particle including nucleic acid of one or more individuals, including the steps of:
a) separation of the particles and application of 2 to 1000, preferably 2 to 100, particularly preferably 2 to 10 and especially preferred 2 to 5 particles onto a hydrophilic reaction site of a substrate in a volume of less than 10 μl with the hydrophilic reaction site being surrounded by a hydrophobic region and
b) investigation of the at least two reaction sites of the substrate with particles deposited on the reaction sites in order to respectively associate the particles by genotyping with individuals from the mixed sample, wherein at least 80% of the investigated particles are associated with one individual, and
c) further characterization of the investigated particles.

Furthermore, the present invention relates to a kit for the determination of the genotype of one or more individuals from a mixed sample which contains particles with nucleic acids of different individuals for the carrying out of the above-described method in accordance with the invention comprising:
a) at least one primer pair which is adapted to amplify in at least one PCR a polymorphous range which is included in at least one of the nucleic acids contained in the mixed sample,
b) a substrate, preferably a glass object carrier, on which at least one, preferably between 2 and 1000 and particularly preferably between 24 and 96 hydrophilic reaction sites surrounded by a hydrophobic region are provided,
c) if required a PCR buffer and
d) a protocol for carrying out of the PCR in accordance with a).

In the sense of the present invention a polymorphous region is understood to be a region from the genome which is distinguished between randomly selected individuals not related to one another with a probability of at least 25%, preferably at least 50%, particularly preferably at least 80% and especially preferably at least 90%, for example in the length of the sequence or in the sequence itself.

In accordance with a preferred embodiment of the present invention the hydrophilic reaction sites provided on the substrate contained in the kit are of essentially circular shape and respectively surrounded by a substantially circular ring-shape hydrophobic region which is concentrically surrounded at the outside by a hydrophilic region which is of substantially circular ring-shape, with the diameter of the hydrophilic reaction sites amounting to between 0.3 and 3 mm. The outer hydrophilic circular ring is preferably surrounded at the outside by a hydrophobic region.

As an option the kit in accordance with the invention can also include one of the following components in addition to the components a), b), d) and optionally c):
e₁) a reference sample with a known genotype and preferably with a copy number known with respect to the predetermined sequence and/or
e₂) the result of at least one amplification reaction carried out under the same conditions as prescribed in the protocol in accordance with e) with a reference sample, with the reaction conditions being so selected that at least one amplification product arose with a probability between 20% and less than 100% and/or
e₃) at least one frequency distribution which was obtained by a separate in each multiple carrying out of the same at least one amplification reaction and under the same reaction conditions as prescribed in the protocol e) with at least two different reference samples, with the at least two different reference samples each having a known copy number of a predetermined sequence different from one another and also subsequent determination of the number of different amplification products obtained in the reference sample.

In the following the present invention will be explained with reference to examples which explain it but do not restrict it:

### EXAMPLE 1

The object of the following investigation was the quantitative relative determination of the number of healthy cells and cancerous cells of a person containing cancerous cells included in a mixed sample.

For the investigations a glass object carrier was used as a substrate on which 48 circular hydrophilic reaction sites spatially separated from one another were arranged, each being concentrically surrounded, considered from the inside towards the outside by a circular hydrophobic region and an adjoining circular ring-shaped hydrophilic region.

For the determination cells from the mixed sample, i.e. from the cancerous tissue, were singled out using a Laser Capture microscope, and in each case one cell taken at random from the mixed sample was deposited in a volume of less than 1 μl on the 48 hydrophilic reaction sites of the substrate. Thereafter a reaction solution was supplied to each reaction site containing a primer pair which amplifies the gene section D85522, a reaction buffer and Taq-polymerase, so that the total volume of the liquid present on each reaction site amounted to 1 μl. Thereafter the individual liquid drops were coated over with oil, the substrate was transferred into a PCR thermo cycler and a PCR was carried out. Finally, an aliquot was taken from each liquid drop, this was applied to a gel and the amplification products contained in the aliquots were electrophoretically separated by gel electrophoresis and the individual DNA bands were visualized.

Whereas a healthy heterozygote cell contains two alleles of the gene section D85522, an LOH cancer cell only has one such allele, because the allele has been lost by deletion (“loss of heterozygosity”).

The gel electrophoresis showed that 12 of the investigated 48 cells in the PCR only resulted in one amplification product and could consequently be associated with the LOH cancer cells, whereas the other 36 samples in the PCR resulted in two amplification products. Consequently the relative frequency of cancerous cells in the tissue amounted to 25%.

### EXAMPLE 2

It should be examined whether a present biological sample is a sample with female and male cells and, if so, how large the proportion of the female cells in the mixed sample is.

A random sample of the cell suspension was sorted by an FACS sorter of the company DAKO and in each case one of the cells was deposited on the 48 reaction sites of the substrate described in example 1.

Thereafter a reaction solution was supplied to each reaction site containing the specific primer pairs for male and female cells, reaction buffer and Taq polymerase.

In this arrangement in each case 2 pmol of five primer pairs was used which were adapted to amplify in a multiplex PCR five different PCR fragments of human male DNA (type XY) or 4 different PCR fragments of human female DNA (type XX). These were the following primers:

| Primer name | Length of the amplified gene section |
|---|---|
| ChrX-STR5589-CX1 | 243 biphasic pulses |
| ChrX-STR861-CX2 | 312 biphasic pulses |
| ChrX-STR3309-CX3 | 346 biphasic pulses |
| ChrX-STR6285-CX5 | 467 bp |
| ChrY-STR596-CY2 | 604 biphasic pulses |

In total the reaction solution contained the following distinct contents:

| | End | 1 | 50 |
|---|---|---|---|
| Component | concentration | Reaction | Reactions |
| 2× QIAGEN Multiplex PCR | 1× | 0.5 μL | 25 μL |
| Master Mix | | | |
| 5× Q-Solution | 0.7× | 0.14 μL | 7 μL |
| Primer | 0.2 pmol | 0.1 μL | 5 μL |
| Template: individual cell | 1×/reaction | 0.1 μL | 5 μL |
| AdvaGold PCR dye | 1× | 0.1 μL | 5 μL |
| ddH₂O | | 0.06 μL | 3 μL |

In each case 1 μl of this reaction solution was pipetted onto the individual reaction sites. Thereafter the individual liquid drops were coated over with oil, the substrate was transferred into a PCR thermocycler and the PCR was carried out with the following temperature control:

| | |
|---|---|
| 94° C. 10 Min. | |
| 94° C. 30 Sec. | 35 cycles |
| 64° C. 60 Sec. | |
| 72° C. 60 Sec. | |
| 72° C. 10 Min. | |

After the PCR 4 μl “6× loading dye” (MBI Fermentas) was added to each liquid drop then 3 μl of the so obtained PCR/dye mixture was applied to an 8% PAA-TBE gel and subjected to electrophoresis under the usual electrophoresis conditions. A 100 by ladder from Promega was used as the standard. Thereafter the gel was coloured with ethidium bromide and the number of different amplification products was determined for each individual sample.

This showed that 2 of the 48 samples were male cells whereas the remaining 46 samples were female cells. The proportion of the female cells in the mixed sample accordingly amounted to 96%.

### EXAMPLE 3

It should be determined in one method step whether cells with the genotype Trisomie 21 are present in the example.

Whereas healthy body cells are diploid, i.e. have 2 copies of chromosome 21 corresponding Trisomie cells have 3 copies of the chromosome 21.

For the carrying out of the investigation 48 individual cells from the mixed sample were respectively deposited on one reaction site each of a substrate as described in example 1 and subjected to a multiplex PCR. Primer pairs were used which amplify 20 specific PCR products for chromosome 21.

The following result was obtained (number of investigated cells: 48):

| Number of the detected bands | Number of the cases (for 48) |
|---|---|
| 0 | 0 |
| 1 | 4 |
| 2 | 5 |
| 3 | 10 |
| 4 | 21 |
| 5 | 6 |
| 6 | 0 |
| 7 | 0 |
| 8 | 0 |
| 9 | 0 |
| 10 | 0 |
| 11 | 0 |
| 12 | 0 |
| 13 | 0 |
| 14 | 0 |
| 15 | 1 |
| 16 | 0 |
| 17 | 0 |
| 18 | 1 |
| 19 | 0 |
| 20 | 0 |

The values obtained were compared with a frequency table in which the previous named PCR was multiply carried out under the same conditions with two different reference samples namely one of a healthy cell having two copies of a chromosome 21 and one with a Trisomie 21 cell and also the number of the amplification products obtained in each determination was determined and prepared in the form of a frequency table.

A comparison with the frequency table showed that in the investigated mixed sample 2 cells were contained which have a Trisomie 21 namely those samples which resulted in 15 amplification products and 17 amplification products in the PCR whereas the other 46 samples only contained two copies of the chromosome 21.

1-38. (canceled)

39. A method of the characterization of a mixed sample containing at least two particles with nucleic acids of different individuals, wherein each particle nucleic acid includes one or more individuals, including the steps:
a) separating the particles and the application of at least two individual particles onto a substrate, with each of the at least two particles being respectively individually deposited onto a hydrophilic reaction site of the substrate in a volume of less than 10 μl, with each hydrophilic reaction site being surrounded by a hydrophobic region, so that precisely one particle is present with each reaction site,
b) investigating of at least two of the particles deposited on the substrate on the reaction site of the substrate by means of an amplification reaction in order to respectively associate the particles with individuals from the mixed sample by genotyping wherein at least 80% of the investigated particles of an individual are associated with the different individuals and
c) further characterization of the investigated particles.

40. Method in accordance with claim 39, wherein
the characterization of the investigated particles in accordance with step c) is the determination of the absolute and/or the relative number of particles present in the example with the nucleic acid of an individual and/or a genotyping.

41. Method in accordance with claim 39, wherein
the at least two particles deposited on the reaction site of the substrate are cells, preferably unlysated cells or preferably magnetic particles with nucleic acid bound thereon.

42. A method in accordance with claim 39, wherein
the subject is an object carrier, preferably a glass object carrier.

43. A method in accordance with claim 39, wherein
the hydrophilic reaction sites on the substrate are of circular shape and is/are surrounded by a circular ring-like hydrophobic region, preferably concentrically.

44. A method in accordance with claim 39, wherein
the hydrophobic regions surrounding the hydrophilic reaction site is surrounded at the outer side of the substrate by a hydrophilic region which is preferably of circular ring shape and which surrounds the hydrophobic region and particularly preferably concentrically surrounds it with the outer hydrophilic region surrounded at the outer side by a hydrophobic region.

45. A method in accordance with claim 39, wherein
the mixed sample is enriched prior to the separation and prior to the application onto the substrate in accordance with step a) with respect to particles with the nucleic acid of the individual to be investigated, wherein the enrichment preferably takes place by means of fluorescence marked anti-bodies which specifically bind to the particle type to be enriched and so marked, by means of coated catcher particles or coated magnet particles or by means of a through-flow cytometer, preferably by means of a fluorescence activated cell sorter (FACS).

46. A method in accordance with claim 39, wherein
the mixed sample includes maternal blood containing foetal cells or preferably consists of maternal blood containing foetal cells.

47. A method in accordance with claim 39, wherein
the mixed sample is a mixture of healthy cells and cancerous cells which have LOH.

48. A method in accordance with claim 39, wherein
the investigation of the at least two particles by genotyping in accordance with step b) and/or the further characterization of the investigated particles in accordance with step c) takes place on the reaction site of the substrate by an amplification reaction, with the reaction volume of the amplification reactor preferably amounting to less than 10 μl and wherein, prior to the amplification reaction for the investigation of the at least two particles of the purpose of the association to an individual containing an example in accordance with step b) and/or for the characterization of the investigated particles in accordance with step c) the nucleic acid contained in or on the at least two deposited particles is propagated by a non-specific PCR.

49. A method in accordance with claim 48, wherein
the reaction components necessary for carrying out the amplification reaction preferably the primers are deposited on the hydrophilic reaction sites before the at least two particles are deposited on the reaction sites.

50. A method in accordance with claim 48, wherein
for the characterization of the amplification products at least one amplification reaction is carried under the same conditions as those used for the at least two particles deposited from the mixed sample on the reaction sites with a reference sample which preferably has the same quantity of nucleic acid as the deposited particles and which preferably has a known genotype and the number of the different amplification products obtained with this at least one amplification reaction is compared with the number of different amplification products obtained with the amplification reactions carried out with the deposited particles.

51. A method in accordance with claim 48, wherein
the number of different amplification products obtained with the amplification reactions carried out with the at least three deposited particles is compared with at least one frequency distribution which was or is obtained by separate in each case multiple carrying out of the same amplification reactions and under the same reaction conditions as used for the particles deposited from the example on the reaction sites, with the same quantity of nucleic acid as is contained in the particles having used or being used in the amplification reactions, with at least two different reference samples, with the at least two different reference samples respectively having a known copy number of a predetermined sequence different from one another, and also a subsequent determination of the number of different amplification products contained per reference sample.

52. A method for the characterization of a mixed sample containing at least two particles with nucleic acids of different individuals, which each particle including nucleic acid of one or more individuals, including the steps:
a) separating the particles and application of 2 to 1000, preferably 2 to 100, and particularly preferably 2 to 10 and especially preferred 2 to 5 particles onto a hydrophilic reaction site of a substrate the volume of less than 10 μl of the hydrophilic reaction site being surrounded by a hydrophobic region and
b) investigation of the at least two reaction sites of the substrate with the particles deposited on the reaction sites by means of an amplification reaction in order to respectively associate the particles by genotyping with individuals from the mixed sample, wherein at least 80% of the investigated particles are associated to one individual, and
c) further characterization of the investigated particles.

53. A kit for carrying out a method of the characterization of a mixed sample containing at least two particles with nucleic acids of different individuals, wherein each particle nucleic acid includes one or more individuals, including the steps:
a) separating the particles and the application of at least two individual particles onto a substrate, with each of the at least two particles being respectively individually deposited onto a hydrophilic reaction site of the substrate in a volume of less than 10 μl, with each hydrophilic reaction site being surrounded by a hydrophobic region, so that precisely one particle is present with each reaction site,
b) investigating of at least two of the particles deposited on the substrate on the reaction site of the substrate by means of an amplification reaction in order to respectively associate the particles with individuals from the mixed sample by genotyping wherein at least 80% of the investigated particles of an individual are associated with the different individuals and
c) further characterization of the investigated particles, including:
i) at least one primer pair which is adapted to amplify an at least one PCR a polymorphous range which includes at least one of the nucleic acids contained in the mixed sample,
ii) a substrate on which at least two, preferably between 2 and 1000 and particularly preferably between 24 and 96 hydrophilic reaction sites respectively surrounded by a hydrophobic region are provided, with the hydrophilic reaction sites on the substrate being of circular shape and being respectively concentrically surrounded by a circular ring region which is concentrically surrounded at the outer side by a hydrophilic region of circular ring shape, with the diameter of the hydrophilic reaction sites amounting to between 0.3 and 3 mm,
iii) if required a PCR buffer and
iv) a protocol for carrying out the PCR in accordance with i).
